(19) United States
(12) Patent Application Publication
(10) Pub. No.: US 2017/0304094 A1
(43) Pub. Date: Oct. 26, 2017

(54) STENT AND METHOD FOR PRODUCING STENT

(71) Applicant: TERUMO KABUSHIKI KAISHA, Tokyo (JP)

(72) Inventors: Takashi KUMAZAWA, Fujinomiya-city (JP); Takayuki KITOU, Hiratsuka-city (JP)

(73) Assignee: TERUMO KABUSHIKI KAISHA, Tokyo (JP)

(21) Appl. No.: 15/633,015

(22) Filed: Jun. 26, 2017

Related U.S. Application Data
(63) Continuation of application No. PCT/JP2015/082964, filed on Nov. 24, 2015.

(30) Foreign Application Priority Data
Dec. 26, 2014 (JP) 2014-265567

(51) Int. Cl. A61F 2/915

(52) U.S. Cl. CPC A61F 2/915; A61F 2230/0069; A61F 2240/002

(57) ABSTRACT

A stent that includes a stent body, which includes a strut formed from a cylindrical base material. The strut possesses a manufactured minimum outer diameter when the strut is formed from the cylindrical base material. The stent body is configured to be compressed onto an outer surface of an expandable member of a catheter. The stent body is configured to be expanded radially outward by applying a nominal pressure directed by a manufacturer within the expandable member. The strut possesses an expanded outer diameter when the stent body is expanded by the expandable member at a pressure which is two atm lower than the nominal pressure. The manufactured minimum outer diameter of the strut is equal to or larger than expanded outer diameter of the strut when the stent body is expanded by the expandable member at the pressure which is two atm lower than the nominal pressure.

### CROSS-REFERENCES TO RELATED APPLICATIONS

This application is a continuation of International Application No. PCT/JP2015/082964 filed on Nov. 24, 2015, and claims priority to Japanese Patent Application No. 2014-265567 filed on Dec. 26, 2014, the entire content of both of which is incorporated herein by reference.

### TECHNICAL FIELD

The present invention generally relates to a stent functioning as a medical device and a manufacturing method of a stent.

### BACKGROUND DISCUSSION

A stent is a medical device used to treat various diseases caused by a stenosed or occluded lumen of a blood vessel. A stent is used for securing a cavity by widening the stenosed or occluded site. As an example of a stent used in the related art, a known stent is manufactured by processing a metal pipe having a cylindrical shape or is manufactured by molding a polymer material as a main component. Depending on the intended function and an indwelling method, the stent is classified as a balloon expandable stent (such as the stent disclosed in Japanese Patent Application Publication No. 2014-111157) or a self-expandable stent.

In a case of the balloon expandable stent, the stent itself is not provided with a self-expandable function (i.e., the stent is not self-expanding and does not self-expand). When the balloon expandable stent is used, the stent is crimped or compressed (i.e., the outer diameter is reduced) and mounted on an outer surface of a balloon that is in a deflated state. The stent together with the balloon is delivered to a target site (i.e., while the balloon is deflated and the stent is compressed). Once the stent and the balloon reach the target site, the balloon is dilated (inflated) from an inner side of the stent, thereby expanding the outer diameter of the stent. The stent whose outer diameter expands closely sticks to (contacts) an inner surface of a body lumen in a living body, and widens a cavity. Thereafter, the stent is caused to indwell the body lumen in a state where the stent widens the cavity to a constant size for a predetermined period.

### SUMMARY

As described above, the balloon expandable stent is retained on the balloon in a state where the balloon outer diameter is reduced after the stent is manufactured. After the stent is introduced into a living body, the outer diameter of the balloon is expanded by the dilated balloon. If the dilated balloon is then deflated and removed from the living body, there is a phenomenon in which the stent that possesses the expanded outer diameter (i.e., deformed radially outward with diameter expansion) restores its original state due to elastic deformation. The outer diameter of the stent is thereby reduced again. In this way, recoil occurs in some cases. When this recoil remarkably occurs (i.e., when a recoil rate is relatively high), a radial force applied to an inner surface of the body lumen from the stent is weakened as a result. Accordingly, the stent is less likely to stably indwell at the desired position.

When the balloon expandable stent is manufactured in the related art, a relatively small diameter member is used for a base material configuring the stent in order to improve the material yield while a strut is processed. A diameter (outer diameter or inner diameter) of the stent after the diameter expands (that is, the diameter of the stent when the stent is caused to indwell the lumen) depends on the lumen serving as the application target. In other words, the inner and outer diameters of the stent are determined and selected based on the diameter of the target body lumen. However, in general, the outer diameter of the stent after expansion is set to be a dimension larger than the outer diameter of the base material. If the outer diameter of the stent after the outer diameter of the stent expands is set to be the larger dimension than the diameter of the base material, a contraction force (diameter reduction force) is applied to the stent after the diameter expands to restore the diameter of the base material. Therefore, the balloon expandable stent in the related art is deformed with diameter reduction (i.e., deflated radially inward to possess a smaller outer diameter) at a relatively high recoil rate.

The stent disclosed in this application possesses a decreased recoil rate. The manufacturing method of a stent disclosed in this application results in a stent having a relatively lower recoil rate.

The stent includes a stent body configured so that a strut is formed in a base material having a cylindrical shape, and is deformed with diameter expansion by expansion of an expandable member. The strut has at least a linear portion in which an axially orthogonal cross section of an outer surface and/or an inner surface forms an arc. A minimum diameter within a diameter of a virtual circle obtained by each of the arcs is equal to or larger than an expanded diameter of the stent body when the expandable member is expanded at pressure which is 2 atm lower than nominal pressure so as to deform the stent with diameter expansion. In another aspect, a stent includes a stent body which has a strut formed from a cylindrical base material. The strut possesses a manufactured minimum outer diameter when the strut is formed from the cylindrical base material. The stent body is configured to be compressed onto an outer surface of an expandable member of a catheter. The stent body is configured to be expanded radially outward by applying a nominal pressure directed by a manufacturer within the expandable member. The strut possesses an expanded outer diameter when the stent body is expanded by the expandable member at a pressure which is 2 atm lower than the nominal pressure. The manufactured minimum outer diameter of the strut is equal to or larger than expanded outer diameter of the strut when the stent body is expanded by the expandable member at the pressure which is 2 atm lower than the nominal pressure.

The diameter (outer diameter, inner diameter, or both of these) of the base material of the stent (i.e., the stent at the time of manufacturing) is equal to or larger than the diameter (outer diameter, inner diameter, or both of these) of the stent body when the stent starts to be used (when the outer diameter of the stent body is expanded so that the stent indwells in a body lumen). Accordingly, it is possible to restrain a contraction force (diameter reduction force) generated when the stent body contracts (i.e., decreases in outer diameter) after having the outer diameter of the stent body expanded in the body lumen (i.e., the contraction force is lower than when the stent body in the expanded outer diameter state is larger dimension than the outer diameter of the base material at the time that the stent is manufactured). Therefore, it is possible to considerably decrease a recoil rate.

Also disclosed in this application is a stent manufacturing method that includes forming a stent body by processing a base material having a cylindrical shape. The stent body includes a strut and is configured to be compressed onto an outer surface of an expandable member of a catheter. The stent body is configured to be positioned in a living body while being compressed on the outer surface of the expandable member and expanded radially outward by applying a nominal pressure directed by a manufacturer within the expandable member. The strut possesses an expanded outer diameter when the stent body is expanded in the living body by the expandable member at a pressure which is 2 atm lower than the nominal pressure. The outer diameter of the base material is equal to or larger than the expanded outer diameter of the strut when the expandable member is expanded at the pressure which is 2 atm lower than nominal pressure so as to radially outwardly expand the stent.

In another aspect, the stent manufacturing method includes determining a target outer diameter for a stent based on a diameter of a target body lumen in a living body, selecting a cylindrical base material with an outer diameter larger than the target outer diameter for the stent, and forming a stent body by cutting a strut out of the cylindrical base material. The stent body is configured to be compressed onto an outer surface of an expandable member of a catheter. The stent body is further configured to be positioned in the living body while being compressed on the outer surface of the expandable member and moved to the target body lumen and then expanded radially outward by applying a nominal pressure directed by a manufacturer within the expandable member. The strut possesses an expanded outer diameter when the stent body is expanded in the living body by the expandable member at a pressure which is 2 atm lower than the nominal pressure. The outer diameter of the cylindrical base material is equal to or larger than the expanded outer diameter of the strut when the expandable member is expanded at the pressure which is 2 atm lower than nominal pressure so as to radially outwardly expand the stent.

### BRIEF DESCRIPTION OF THE DRAWINGS

FIGS. 1(A) and 1(B) are views illustrating one embodiment of a stent. FIG. 1(A) is a development view of the stent, and FIG. 1(B) is an enlarged view of a portion indicated by a broken line portion 1B in FIG. 1(A).

FIGS. 2(A) and 2(B) are views illustrating a base material serving as a configuration member of the stent illustrated in FIG. 1(A). FIG. 2(A) is a schematic perspective view of the base material, and FIG. 2(B) is a view schematically illustrating an axially orthogonal cross section taken along line 2B-2B illustrated in FIG. 2(A).

FIGS. 3(A) and 3(B) are views illustrating a stent body included in the stent according to the embodiment. FIG. 3(A) is a schematic perspective view of the stent body, and FIG. 3(B) is a view schematically illustrating an axially orthogonal cross section taken along line 3B-3B illustrated in FIG. 3(A).

FIGS. 4(A) and 4(B) are views illustrating the stent being crimped. FIG. 4(A) is a schematic perspective view of the stent when the stent is crimped, and FIG. 4(B) is a view schematically illustrating an axially orthogonal cross section taken along line 4B-4B illustrated in FIG. 4(A).

FIGS. 5(A) and 5(B) are views illustrating the stent being deformed with the outer diameter of the stent expanding radially outward by the dilation of a balloon. FIG. 5(A) is a schematic perspective view of the stent when the stent is deformed with the diameter expansion (i.e., expanded radially outward to possess a larger outer diameter), and FIG. 5(B) is a view schematically illustrating an axially orthogonal cross section taken along line 5B-5B illustrated in FIG. 5(A).

FIGS. 6(A) and 6(B) are views for describing a principle for measuring an outer diameter of the base material configuring the stent. FIG. 6(A) is a perspective enlarged view of a portion of a strut, and FIG. 6(B) is an enlarged view of a portion of the axially orthogonal cross section of the strut.

FIG. 7 is a view illustrating a change in an outer diameter of the stent body according to an application example.

Set forth below with reference to the accompanying drawings is a detailed description of embodiments of a stent and a stent manufacturing method representing examples of the inventive stent and manufacturing method disclosed here. The following description does not limit the technical scope or the meaning of terms described in the scope of the appended claims. Dimensional proportions in the drawings are exaggerated and different from actual proportions for convenience of description.

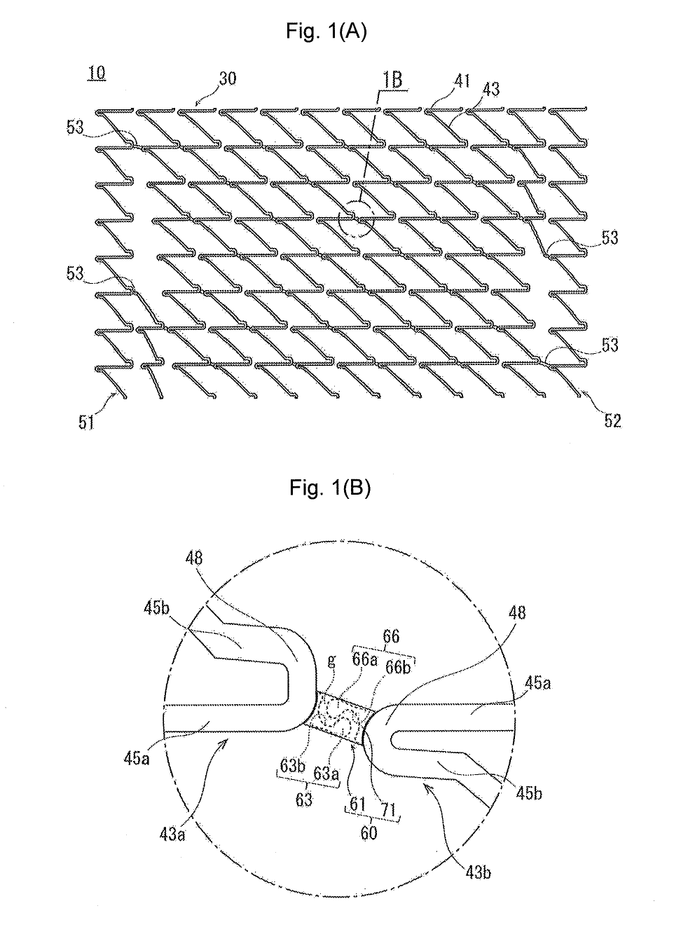

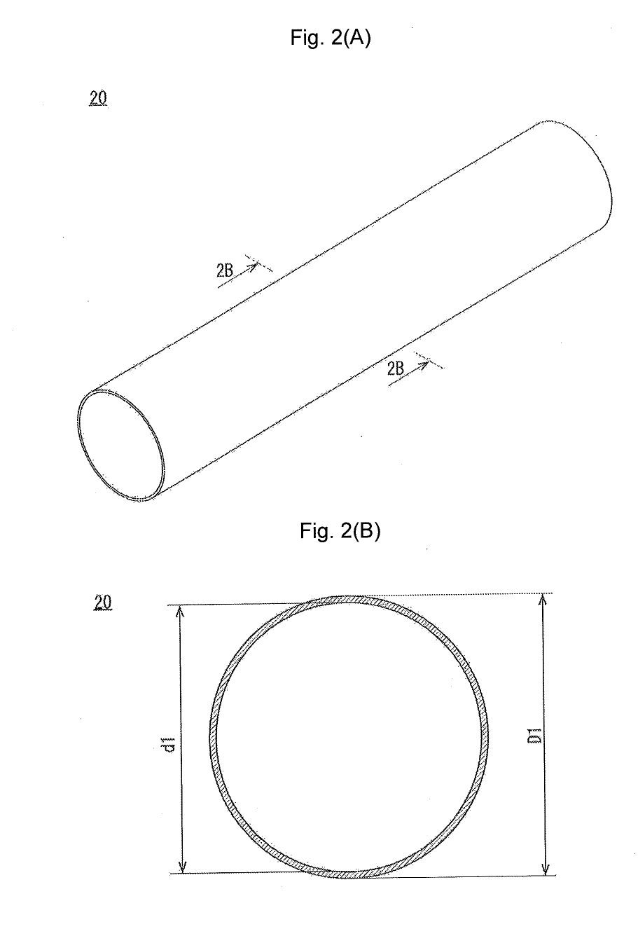

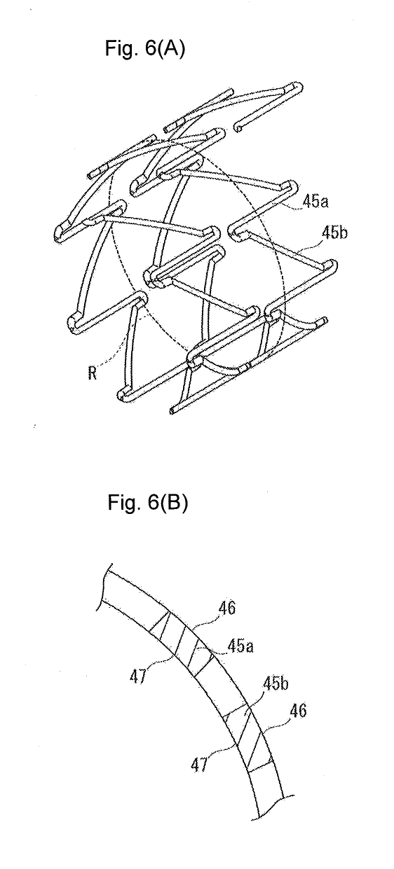

FIGS. 1(A) and 1(B) are views that illustrate a configuration of one embodiment of a stent. FIGS. 2(A) to 5(B) are views that show the relationship between the outer diameters of the stent, a stent body, and a base material. FIGS. 6(A) and 6(B) are views for describing a principle for measuring the outer diameter of the base material. In the description below, a longitudinal direction (lateral direction in FIG. 1(A)) of the stent is referred to as the axial direction.

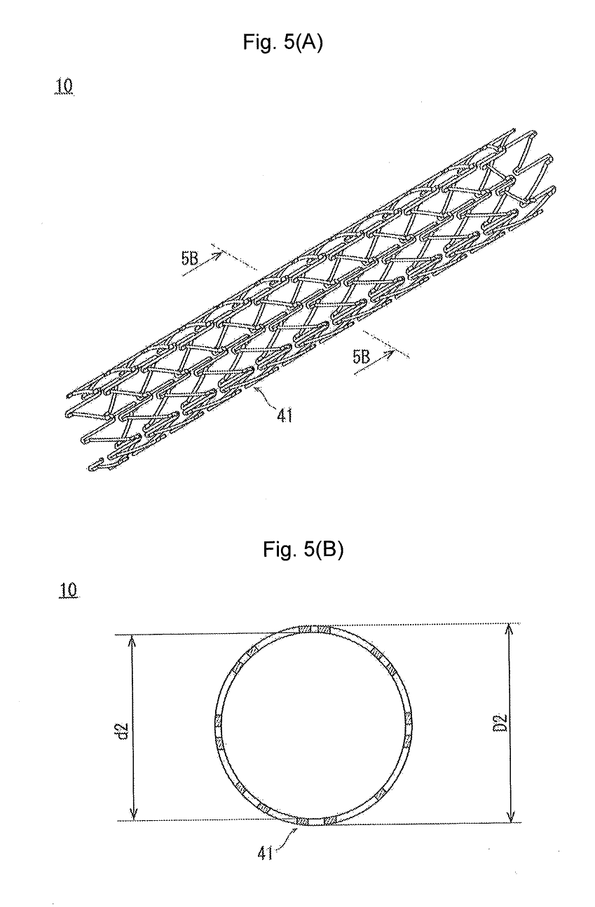

As illustrated in FIG. 1(A), one embodiment of a stent 10 includes a stent body 30 in which a strut (linear configuration element) 41 having an integrally connected coil shape is formed to possess a cylindrical shape. As a whole, the stent 10 is formed to possess a substantially cylindrical outer shape having a predetermined length in the axial direction (e.g., see FIG. 5(A)). The stent 10 is caused to indwell a lumen in a living body (for example, a blood vessel, a biliary duct, a bronchial tube, an esophagus, other gastrointestinal tracts, and a urethra) inside a living body. The stent 10 is used for treating a stenosed or occluded site by widening a cavity of the body lumen. In addition, the stent 10 is a so-called balloon expandable stent (i.e., a balloon expanding-type stent) which is caused to indwell the lumen after being deformed with the outer diameter of the stent 10 expanding by the inflation of a balloon (corresponding to an “expandable member”) included in a balloon catheter.

As illustrated in FIG. 1(A), the strut 41 has a plurality of helical portions 43 which extend in a helical shape in the circumferential direction (i.e., around the axial direction) of a stent body 30 while being turned back in a wavelike manner in the axial direction (longitudinal direction) of the stent body 30. The strut 41 also includes endless annular portions 51 and 52 arranged in both end portions in the axial direction of the stent body 30 (i.e., at the distal end and the proximal end of the stent body 30).

The stent body 30 includes the helical portions 43 and the annular portions 51 and 52 that are formed integrally. Adjacent helical portions 43 are connected to each other via a connection portion 60. Each of the respective annular portions 51 and 52 are connected to the adjacent helical portions 43 via a link portion 53. The link portion 53 together with the helical portions 43 and the annular portions 51 and 52 are formed integrally to form at least a portion of the stent body 30.

As illustrated in FIG. 1(B), the helical portion 43 included in the strut 41 has a pair of linear portions 45a and 45b extending in the axial direction of the stent body 30, and a curved portion (turned-back portion) 48 disposed between the pair of linear portions 45a and 45b. The linear portions 45a and 45b and the curved portion 48 are repeatedly formed along a predetermined length, thereby configuring one helical portion 43. The helical portions 43 are disposed in series in a side by side manner along the axial direction of the stent body 30, thereby allowing the overall stent 10 to configure one helix (i.e., the stent 10 is helix-shaped or formed in the shape of a helix). The number of the helical portions 43 and the number of the curved portions 48 is not particularly limited. In the development view of the stent 10, the linear portions 45a and 45b refer to portions of the stent 10 whose outer shapes are illustrated in a substantially linear shape. An angle of the linear portions 45a and 45b extending with respect to the axial direction of the stent 10 and a specific shape of the linear portions 45a and 45b are not limited to those which are illustrated.

As illustrated in FIG. 1(B), the connection portion 60 has a connection structure 61 formed integrally with the helical portion 43 of the strut 41. The connection portion 60 includes a connection member 71, which is configured to include a biodegradable material.

The connection structure 61 is formed by adding a predetermined shape to the pair of helical portions 43a and 43b arranged adjacent to one another so that the helical portions 43a and 43b face each other in the axial direction. In the example illustrated in FIG. 1(B), the connection structure 61 is configured to include a first engagement portion 63 formed in one adjacent helical portion (hereinafter, referred to as a “first helical portion”) 43a, and a second engagement portion 66 formed in the other helical portion (hereinafter, referred to as a “second helical portion”) 43b. The first engagement portion 63 and the second engagement portion 66 are engaged with (hooked to) each other, thereby mechanically connecting the helical portions 43a and 43b to each other.

The first engagement portion 63 includes a first protruding portion 63a that protrudes toward the second helical portion 43b side from the curved portion 48. The first engagement portion 63 also includes a first housing portion 63b formed to be recessed in a concave shape between the first protruding portion 63a and the curved portion 48. In addition, the second engagement portion 66 includes a second protruding portion 66a that protrudes toward the first helical portion 43a side from the curved portion 48 and a second housing portion 66b formed to be recessed in a concave shape between the second protruding portion 66a and the curved portion 48.

The first protruding portion 63a included in the first engagement portion 63 is formed so that the distal portion of the first engagement portion 63 is curved. The second housing portion 66b included in the second engagement portion 66 is formed so that the first protruding portion 63a can be housed within (i.e., positioned in) the second housing portion 66b. The second protruding portion 66a included in the second engagement portion 66 also possesses a curved distal portion as shown in FIG. 1(B). The first housing portion 63b included in the first engagement portion 63 is formed so that the second protruding portion 66a can be housed within (i.e., positioned in) the second housing portion 66b. When the first protruding portion 63a is housed inside the second housing portion 66b and the second protruding portion 66a is housed inside the first housing portion 63b, the first helical portion 43a and the second helical portion 43b (which are adjacent to each other) are mechanically connected via the first engagement portion 63 and the second engagement portion 66.

The respective protruding portions 63a and 66a can be arranged so as to form a gap g between the respective housing portions 63b and 66b as illustrated in FIG. 1(B). The protruding portions 63a and 63b can also be arranged so as to partially come into contact with the respective housing portions 63b and 66b. The respective engagement portions 63 and 66 can additionally be arranged so that both of the engagement portions 63 and 66 partially or entirely overlap each other in the circumferential direction and/or the axial direction of the stent 10 as illustrated in FIG. 1(B). According to this arrangement with the engagement portions 63 and 66 circumferentially and axially overlapping, it is possible to strongly hook the respective engagement portions 63 and 66 to each other. It is thus possible to stably maintain a connection state between the first engagement portion 63 and the second engagement portion 66. In addition, as illustrated in FIG. 1(B), it is possible to arrange the respective protruding portions 63a and 66a to face each other in a direction tilting with respect to the axial direction. In other words, the respective protruding portions 63a and 66a each extend at an angle relative to the axial direction. If both of protruding portions 63a and 66a are arranged in this way, when a tensile force is applied to separate the first helical portion 43a and the second helical portion 43b along the axial direction, a distance between the respective protruding portions 63a and 66a is narrowed, thereby attaching the protruding portions 63a and 66a to each other. The first engagement portion 63 and the second engagement portion 66 are thus strongly hooked (i.e., mechanically fastened or connected) to each other. It is thus possible to more reliably maintain a connection state between the helical portions 43a and 43b.

The connection member 71 is disposed to cover a surface of the connection structure 61 and to fill portions between the respective protruding portions 63a and 66a and the respective housing portions 63b and 66b. A configuration can be adopted in which a concave portion is formed or a through-hole penetrating both front and back surfaces is formed on the surface of the respective engagement portions 63 and 66. The connection member 71 may thus fill the concave portion or the through-hole. According to this configuration, it is possible to improve adhesion (i.e., the adhesive force) of the connection member 71 adhering to the connection structure 61.

Since the stent 10 in the FIG. 1(A) embodiment includes the helical portion 43, the stent 10 is flexible. Therefore, the stent's ability to follow the deformation of the lumen (followability) is improved. The connection member 71 formed of the biodegradable material having a relatively strong physical property (i.e., the biodegradable material applies a relatively strong adhesion/boding force to connect the helical portions 111 to each other) is disposed in a portion connecting the helical portions 43 to each other. Accordingly, the stent body 30 can be provided with desirable rigidity. While satisfactory followability to follow the deformation of the lumen is ensured, an expansion holding force can be improved when the stent 10 is caused to indwell the lumen. The indwelling connection member 71 degrades after a predetermined period elapses so that the connecting force of the connection portion 60 weakens. When this degradation occurs, flexibility of the stent 10 is further improved. Accordingly, the followability of the stent to follow the deformation of the lumen is further improved. Therefore, in an initial stage of the indwelling period, a desired expansion holding force is achieved, and after the predetermined period elapses from the indwelling and the connection member 71 degrades, improved flexibility is achieved. The stent 10 thus becomes relatively excellent in regard to invasiveness and a treatment effect. In addition, the annular portions 51 and 52 (which are disposed in both end portions of the stent body 30) maintain a predetermined expansion holding force regardless of the degradation of the connection member 71. Therefore, it is possible to apply a sufficient expansion holding force to the body lumen from both end portions (i.e., the distal and proximal ends) of the stent body 30 even after the connection member 71 degrades. Accordingly, it is possible to suitably prevent the stent 10 from being misaligned after the stent 10 indwells.

It is preferable to provide one or more connection portions 60 for each one of the helical portions 43 (one unit of the helical portion in the circumferential direction). However, the number of connection portions 60 is not particularly limited. The structure of the connection portion 60 and the form of the connection structure 61 and the connection member 71 (which are included in the connection portion 60) are not limited to the above-described configurations. The structure and the form of the connection portion 60 can be appropriately changed. For example, the shape of the respective engagement portions 63 and 66 included in the connection structure 61 can be different than the shapes discussed above as long as a mechanical connection can be created. The connection portion 60 can be configured to change the connecting force without interposing the connection member 71 therebetween. For example, it is possible to employ a fragile portion (which is more likely to be broken than other portions) in a portion of the connection structure 61. The fragile portion breaks after a predetermined period elapses in a state where the stent 10 indwells. In this manner, the connection structure 61 can oscillate (is movable).

Next, a relationship between an outer diameter D1 of the base material 20 serving as a configuration member of the stent 10 and an expansion start outer diameter D2 of the stent body 30 is described.

The expansion start outer diameter (expansion outer diameter or expanded outer diameter) D2 of the stent body 30 represents the outer diameter of the stent body 30 when the stent 10 is caused to indwell the lumen of the living body. That is, the expansion start outer diameter represents the outer diameter of the stent body 30 indwelling when the balloon has been dilated and the stent 10 is thus deformed to possesses the expanded outer diameter. In other words, the expansion start outer diameter is the outer diameter of the stent body after the balloon is dilated and deformed at predetermined pressure in a state where the stent 10 is crimped (compressed) on the outer surface of the balloon. In general, the dilating pressure of the balloon when the stent is used is determined based on (or guided by) a compliance chart. This compliance chart may be in a compliance sheet enclosed when the stent is commercially distributed as a product. The compliance chart provides guidance regarding the relationship between the dilating pressure of the balloon and the inner diameter of the stent. The compliance chart shows the nominal pressure indicating how much pressure is required to dilate the balloon in order to cause the stent to indwell by using a prescribed inner diameter (inner diameter of the product). The stent 10 according to the embodiment of FIG. 1(A) is configured as follows. When the balloon is dilated at a pressure that is 2 atm lower than the nominal pressure (the pressure is obtained by subtracting 2 [atm] from the nominal pressure [atm]; hereinafter, this obtained pressure is referred to as the “dilating start pressure”), the outer diameter (hereinafter, referred to as the “expansion start outer diameter”) D2 of the stent body 30 is equal to or smaller than the outer diameter D1 of the base material 20.

FIGS. 2(A) and 2(B) illustrate the base material 20. The base material 20 is a pipe material having a hollow cylindrical shape. The present embodiment employs a non-biodegradable metal material as the base material 20, but additional materials that may be used as the base material 20 are described below. The base material 20 has a circular cross section having a uniform outer diameter D1 in the axial direction.

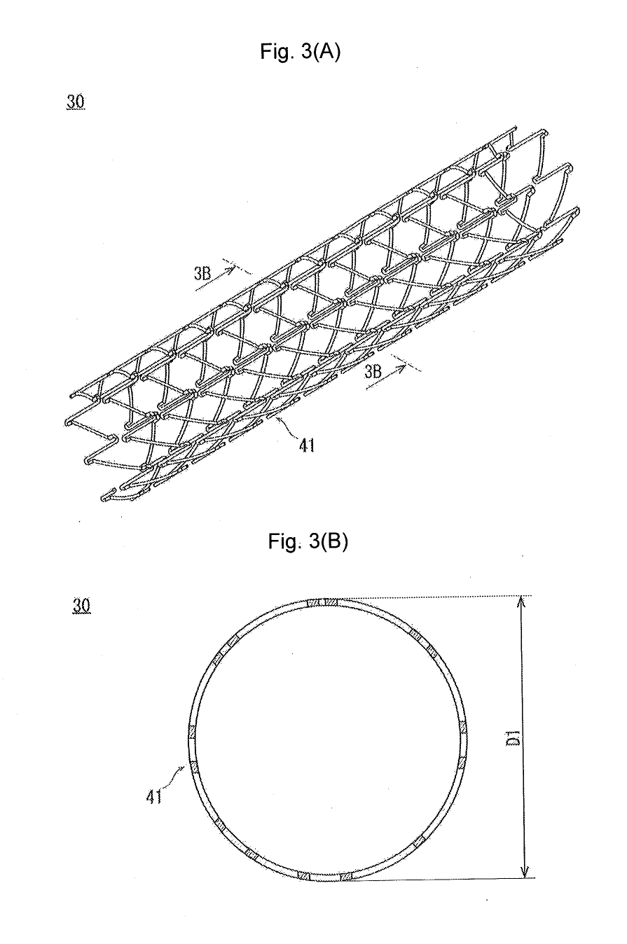

FIGS. 3(A) and 3(B) illustrate the stent body 30. The stent body 30 is in a state in which the strut 41 (i.e., including the helical portion, annular portion, and connection structure) is formed for the base material 20 having the cylindrical shape. The stent body 30 is manufactured by removing a portion of the base material 20 to form a gap portion. The remaining portion, which is not removed from the base material 20, configures the strut 41. The outer diameter of the stent body 30 before the stent body 30 is deformed via diameter expansion (i.e., before use to expand the stent body 30) is substantially the same as the outer diameter of the base material 20. After the stent body 30 is manufactured, the stent 10 is manufactured by processing the stent body 30 through a polishing process, a process of forming the connection portion 60, and a process of forming a drug coated layer.

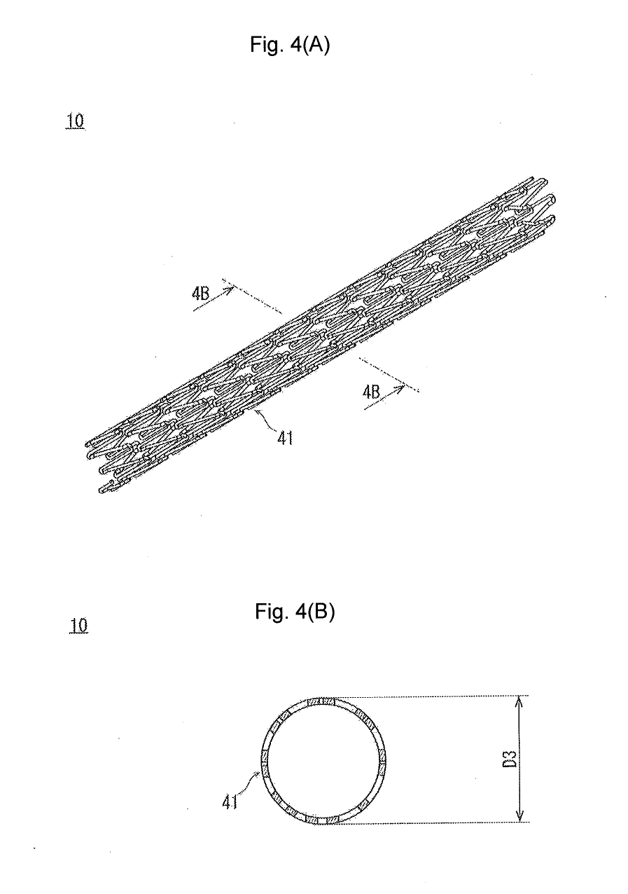

FIGS. 4(A) and 4(B) illustrate the stent 10 when the stent 10 is crimped (i.e., compressed) on the outer surface of the balloon (illustration omitted) of the balloon catheter. Before the stent 10 is introduced into the living body, the stent 10 is crimped on the outer surface of the balloon. The stent 10 is thus prepared in a state where the outer diameter of the stent 10 is reduced. An outer diameter D3 of the stent 10 when the stent 10 is crimped is smaller than the outer diameter D1 of the base material 20 and the expansion start outer diameter D2 (i.e., the outer diameter of the stent 10 when the stent 10 is expanded radially outward by inflating the balloon). For example, it is possible to use a known balloon catheter such as a rapid exchange type and an over-the-wire type as the balloon catheter that delivers the stent 10 into the living body.

FIGS. 5(A) and 5(B) illustrate the stent 10 when the balloon is dilated at the dilating start pressure. As described above, the outer diameter D1 of the base material and the expansion start outer diameter D2 satisfy a relationship of (D1)≧(D2) in the stent 10 illustrated in FIGS. 1(A)-5(B). In manufacturing the stent in the related art, a base material which is as thin as possible is used for reasons such as material cost reduction. The outer diameter D1 of the base material in manufacturing these stents in the related art is thus generally set to be smaller than the expansion start outer diameter D2. After the stent has been deformed to possess the expanded outer diameter, a contraction force to restore the outer diameter D2 of the stent body to the original outer diameter D1 is applied to the stent. This contraction force to restore the outer diameter D2 of the stent body to the original outer diameter D1 causes the recoil to occur very greatly (i.e., there is a relatively large recoil). In contrast, the outer diameter D1 of the base material 20 of the stent 10 illustrated in FIGS. 1(A)-5(B) has a size which is equal to or larger than the expansion start outer diameter D2. Accordingly, it is possible to restrain the recoil from occurring due to the above-described factor.

The following description explains the reason that the outer diameter D2 of the stent body 30 when the stent body 30 is expanded by the balloon at the dilating start pressure (nominal pressure [atm]−2 [atm]) is compared with the outer diameter D1 of the base material 20.

The nominal pressure is only an indication when the stent 10 is deformed with diameter expansion. Accordingly, even if the stent body 30 is configured to have the desired expansion start outer diameter D2 after the outer diameter D1 of the base material 20 is properly selected, the stent body 30 is affected by product dimension fluctuations occurring at the manufacturing stage. When the stent body 30 is expanded at the nominal pressure, the relationship of (D1)≧(D2) may thus not be strictly satisfied. On the other hand, if the stent body 30 is manufactured based on the outer diameter of the stent body 30 when the stent body 30 is deformed with diameter expansion (i.e., expanded radially outward to indwell the body lumen) at a pressure which is 2 atm lower than the nominal pressure, there is very high probability that the relationship of (D1)≧(D2) may be satisfied even if there are some product dimension fluctuations during manufacturing.

In addition, when the relationship of (D1)≧(D2) is satisfied by using a pressure state of 2 atm less than the nominal pressure, a diameter expansion deformation amount (diameter expansion deformation ratio) of the stent body 30 falls within a certain degree range with respect to the outer diameter D1 of the base material 20 even if the relationship of (D1)≧(D2) is not satisfied when the stent 10 is expanded at the nominal pressure. Accordingly, stress (distortion) accumulated in the stent body 30 when the stent body 30 is deformed with diameter expansion (i.e., expanded radially outward to indwell the body lumen) is reduced. In this manner, it is possible to restrain recoil due to stress accumulated in the stent body 30.

The compliance sheet enclosed when the stent 10 is commercially distributed as a product to generally show a change in the inner diameter of the stent (relationship between the pressure applied and the inner diameter of the stent) when the stent 10 is deformed with diameter expansion (i.e., expanded radially outward from the compressed position on the outer surface of the balloon) by applying rated burst pressure (RBP) to the nominal pressure—2 [atm] (i.e., the nominal pressure minus 2 atm) or pressure weaker than this pressure to the stent 10. Therefore, the dimensional characteristics of the stent 10 are defined in advance based on the outer diameter D2 of the stent body 30 when the diameter is expanded at the nominal pressure [atm]−2 [atm] (i.e., expanded at the nominal pressure [atm]minus 2 [atm]). In this manner, the compliance chart providing guidance in the compliance sheet can be used as an index to determine whether or not the operation the same as that of the stent 10 according to the present embodiment can be obtained. Therefore, the stent 10 provides satisfactory usability for a user of the stent. However, the guidance provided by the compliance sheet is only an indication for confirming the inner diameter when the stent 10 has the expansion start outer diameter D2. Accordingly, for example, the actual expansion start outer diameter D2 can be defined by an actual measured value.

Even when the stent 10 is deformed with diameter expansion at the nominal pressure [atm]−1 [atm] (i.e., the stent 10 is expanded radially outward by applying the nominal pressure [atm]minus 1 [atm]), the stent 10 can be configured so that the outer diameter D2 of the stent body 30 is equal to or smaller than the outer diameter D1 of the base material 20. That is, the stent 10 satisfies the relationship of (D1) (D2). It is thus possible to help prevent the relationship of (D1) (D2) from not being satisfied due to an error caused by the product dimension fluctuations, and it is possible to suitably restrain the recoil from occurring when in use. The indicator of the expansion start outer diameter D2 can be confirmed based on the guidance provided by the compliance sheet. These points are similarly applied to a relationship between an inner diameter d1 of the base material 20 and an expansion start inner diameter d2 of the stent body 30 (to be described later).

Next, a measurement method of the outer diameter D1 of the base material 20 is described in reference to FIGS. 6(A) and 6(B).

The outer diameter D1 of the base material 20 can be defined as the known outer diameter when the outer diameter (original diameter) D1 of the base material 20 to be used is already known. On the other hand, when the outer diameter D1 of the base material 20 is not known, the outer diameter D1 of the base material 20 can be measured from a shape of the strut 41 formed in the stent body 30. As described above, the stent body 30 is configured so that the strut 41 is formed from the cylindrically-shaped base material 20. Accordingly, the outer surface 46 of the strut 41 in the stent body 30 has the outer surface shape of the base material 20. As illustrated in FIG. 6(B), an arc (circular arc) formed by the outer surface 46 of the strut 41 on an axially orthogonal cross section of the stent body 30 has a curvature that is substantially the same as that of an arc formed by the outer surface of the base material 20 to be used. A virtual circle R including the arc formed by the outer surface 46 of the strut 41 illustrated in FIG. 6(A) has the same sectional shape as the axially orthogonal cross section of the base material 20. In other words, the virtual circle R includes the arcs of the plurality of outer surfaces 46 of the linear portions 45a and 45b as shown in FIGS. 6(A)-6(B), and so the virtual circle R represents the outer diameter of the strut when the strut is manufactured from the cylindrical base material. Therefore, the diameter of the virtual circle R (and thus the outer diameter D1 of the base material 20) can be obtained by obtaining a radius of the virtual circle R. In FIG. 6(B), a portion of the strut 41 is exaggerated, and a strict sectional image is not illustrated.

When the outer diameter D1 of the base material 20 is obtained (i.e., the circumferential diameter at the outer surface 46 of the strut 41), the outer surface of the linear portions 45a and 45b (refer to FIG. 1(B)) of the strut 41 is selected. The curved portion 48 connected to the linear portions 45a and 45b of the strut 41 is a starting point of the deformation when the stent 10 is deformed (i.e., deformation radially inward with diameter reduction after the stent 10 is manufactured and crimped or deformation radially outward with diameter expansion when the stent 10 is in use in the body lumen). There is a possibility that the outer surface shape of the strut 41 may be deformed due to the diameter reduction and the diameter expansion. Therefore, there is a possibility that the outer diameter D1 of the base material 20 cannot be accurately measured if the outer diameter D1 of the base material 20 is obtained based on the outer surface of the curved portion 48 of the strut 41. In contrast, the linear portions 45a and 45b of the strut 41 are relatively less affected by the diameter reduction and the diameter expansion of the strut 41, and so the cross-sectional shape is less likely to be deformed. The outer diameter D1 of the base material 20 can thus be accurately measured based on measuring the circumferential diameter along the outer surface 46 of the linear portions 45a and 45b.

The linear portions 45a and 45b of the strut 41 are disposed at a plurality of different positions in the circumferential direction and the axial direction. Accordingly, there is a possibility that the shape of the arc of the measured outer surface 46 may vary at each position of the linear portions 45a and 45b. For example, the sectional shape of the outer surface 46 varies at each position of the linear portions 45a and 45b of the strut 41. As a result, it becomes difficult to unmistakably define the outer diameter D1 of the base material 20 if fluctuations occur in the outer diameter D1 of the measured outer surfaces 46 of the linear portions 45a and 45b. In order to avoid this problem, the minimum radius in the respective radii obtained from the outer surface 46 of the linear portions 45a and 45b of the strut 41 can be employed in advance as a representative value. The stent body 30 is configured to include the base material 20 having the cylindrical shape. There is thus a possibility that the strut 41 having a radius larger than the radius of the base material 20 may exist. However, the strut 41 will not have a radius smaller than the radius of the base material 20. Therefore, the minimum diameter (the manufactured minimum outer diameter) of the virtual circle R is calculated based on the minimum radius in the radii obtained from the arc formed by the outer surfaces 46 of the linear portions 45a and 45b of the strut 41. This measurement technique makes it possible to accurately confirm the outer diameter D1 of the base material 20 to be used for the stent 10. The time to measure the outer diameter of the base material 20 from the stent body 30 may be after the outer diameter of the stent 10 has been expanded or after when the stent 10 has been crimped. The sectional shape of the linear portions 45a and 45b of the strut 41 is much less affected by the diameter expansion and the diameter reduction of the stent 10 than the curved portion 48. The linear portions 45a and 45b are to maintain a constant shape.

As described above, the outer diameter D1 of the base material 20 can be obtained from the arc formed by the outer surface 46 of the linear portions 45a and 45b of the strut 41. Similarly, the outer diameter D1 of the base material 20 can also be obtained by measuring the arcs formed by the inner surfaces 47 of the linear portions 45a and 45b of the strut 41. Similarly to the outer surface 46, the inner surface 47 of the strut 41 has an arc-shaped sectional shape corresponding to the inner surface of the used base material 20. The curvature of the arc is the same as the curvature of the cross section of the inner surface of the base material 20. The inner diameter of the base material 20 can thus be obtained based on the shape (i.e., curvature) of the inner surface 47 of the linear portions 45a and 45b of the strut 41. The thickness of the linear portions 45a and 45b of the strut 41 may also be measured, so that it is possible to obtain (i.e., determine or calculate) the outer diameter D1 of the base material 20.

For example, the diameter of the base material 20 and the diameter of the stent body 30 can be compared at both the inner diameters instead of at the outer diameters of the base material 20 and of the stent body 30. When the inner diameter d1 of the base material 20 illustrated in FIG. 2(B) and FIG. 5(B) is compared with the expansion start inner diameter (expansion inner diameter) d2 of the stent body 30, if the stent 10 is configured to satisfy a relationship of (d1) (d2) (similar to when the outer diameter D1 of the base material 20 and the expansion start outer diameter D2 of the stent body 30 satisfy the relationship of (D1) (D2)), it is possible to restrain the contraction force from being applied to the stent body 30 after the stent body 30 is deformed with diameter expansion (i.e., expanded radially outward). Therefore, it is possible to restrain the recoil of the stent body 30. The stent 10 may be configured so that each of the outer diameter D1 and the inner diameter d1 of the base material 20 and each of the expansion start outer diameter D2 and the expansion start inner diameter d2 of the stent body 30 satisfy the two relationships—(D1)≧(D2) and (d1)≧(d2). The inner diameter d1 of the base material 20 can also be obtained (determined) by employing the same method as described above regarding measuring the outer diameter D1, from any one of the arcs formed by the outer surfaces 46 of the linear portions 45a and 45b of the strut 41 and the arcs formed by the inner surfaces 47 of the linear portions 45a and 45b of the strut 41.

For example, it is possible to use a known device such as an electron microscope and a laser measurement device in order to measure the dimensions of each portion of the stent 10.

Next, a material and a dimension example of each portion of the stent 10 is described.

The outer diameter D1 of the base material 20 is preferably 2.1 to 30 mm (i.e., a range from 2.1 mm to 30 mm), and more preferably 3.0 to 20 mm. The inner diameter d1 of the base material 20 is preferably 1.9 mm to 29.8 mm, and more preferably 2.7 mm to 19.8 mm. The thickness of the base material 20 is preferably 0.04 to 1.0 mm, and more preferably 0.06 to 0.5 mm. The axial length of the base material 20 is preferably 5 to 250 mm, and more preferably 8 mm to 200 mm.

Although the dimension of the stent body 30 depends on an indwelling target site, the expansion start outer diameter D2 is preferably 2.1 to 20 mm and the expansion start inner diameter d2 is preferably 1.9 mm to 19.8 mm. A helical pitch (interval between the adjacent helical portions 43) is preferably 0.5 to 3 mm, and more preferably 0.8 to 1.5 mm. The outer diameter of the stent 10 when the stent 10 is crimped on the balloon is preferably 0.8 to 1.3 mm.

It is possible to appropriately select a material of the base material 20 from those which are known to configure a balloon expandable stent. For example, it is possible to use a metal other than a super-elastic alloy used for the self-expandable stent. The “metal other than the super-elastic alloy” described herein can be defined as a metal whose permanent elongation is 2% or greater when in compliance with JIS Z 2241, which is a tensile test that is performed so that tensile stress is loaded and unloaded until total elongation of the metal reaches 3% in a temperature environment of 35° C. Examples of these metals that can be used for the base material 20 include an elastic metal such as stainless steel, a cobalt-based alloy such as a cobalt-chromium alloy (for example, CoCrWNi alloy), and a platinum-chromium alloy (for example, PtFeCrNi alloy) which are non-biodegradable metal materials.

In order to form the strut 41 and the connection structure 61 for the base material 20, it is possible to perform cutting (for example, mechanical polishing and laser cutting), electric discharge machining, or chemical etching. These machining techniques can also be used in combination with each other.

The connection member 71 is formed of a biodegradable material such as a biodegradable polymer material or a biodegradable metal material. For example, the biodegradable polymer material may preferably be a biodegradable synthetic polymer material such as polylactic acid, polyglycolic acid, lactic acid-glycolic acid copolymer, polycaprolactone, lactic acid-caprolactone copolymer, glycolic acid-caprolactone copolymer, and poly-y-glutamic acid, or a biodegradable natural polymer material such as cellulose and collagen. Additionally, for example, it is preferable to use magnesium or zinc as the biodegradable metal material.

When the connection portion 60 is filled and coated with the connection member 71, a coating solution obtained by dissolving the connection member 71 in a solvent is applied by using a pivot, for example. The solvent is evaporated, and the connection member 71 is dried and solidified. The connection portion 60 can thus be formed in this manner.

Although the solvent is not particularly limited, it is possible to use organic solvents such as methanol, ethanol, dioxane, tetrahydrofuran, dimethylformamide, acetonitrile, dimethylsulfoxide, and acetone.

A drug coated layer containing a drug can be formed in the stent 10 of the embodiment shown in FIG. 1(A). For example, the drug coated layer can be disposed on an entire outer surface on a side coming into contact with the lumen of the living body or only on a portion of the outer surface. The drug coated layer may contain a drug carrier for carrying the drug or may be configured to contain the drug without having the drug carrier. The thickness of the drug coated layer is, for example, 1 to 300 μm, and preferably is 3 to 30 μm.

The drug contained in the drug coated layer, for example, may be an anticancer drug, immunosuppressive drug, antibiotic, anti-rheumatic drug, anti-thrombotic drug, HMG-CoA reductase inhibitor, insulin resistance improving drug, ACE inhibitor, calcium antagonist, anti-hyperlipidemic drug, integrin inhibitor, anti-allergic drug, anti-oxidant, GP IIb/IIIa antagonist, retinoid, flavonoid, carotenoid, lipid improving drug, DNA synthesis inhibitor, tyrosine kinase inhibitor, antiplatelet drug, anti-inflammatory drug, biologically-derived material, interferon, and nitric oxide production-promoting substance.

When the stent 10 is configured to treat a stenosed site in the blood vessel, it is preferable that the drug coated layer contains paclitaxel, docetaxel, sirolimus, everolimus, biolimus, or zotarolimus. It is more preferable that the drug coated layer contains sirolimus, everolimus, or biolimus.

It is preferable that the drug carrier is polymer material. It is particularly preferable that the drug carrier is a biodegradable polymer material which degrades inside a living body. After the stent 10 is caused to indwell the lumen of the living body, the biodegradable polymer material that carries the drug degrades. The drug is released by this biodegradation to restrain restenosis in the stent indwelling site. It is possible to use a material that is the same as a material of the above-described connection member 71 for the biodegradable polymer material.

As described above, the stent 10 illustrated in FIG. 1(A) includes at least the strut 41 having the linear portions 45a and 45b in which the axially orthogonal cross section of the outer surfaces 46 and/or the inner surfaces 47 forms an arc. The stent 10 is configured so that the minimum diameter in the diameter of the virtual circle R obtained from each arc is equal to or larger than the expansion start diameter of the stent body 30. The diameter (outer diameter, inner diameter, or both of these) of the base material 20 serving as the configuration member of the stent 10 thus has a size which is equal to or larger than the diameter (outer diameter, inner diameter, or both of these) of the stent body 30 when the stent 10 starts to be used (when the stent 10 is caused to indwell in a body lumen with the outer diameter of the stent 10 being expanded). Accordingly, it is possible to restrain (reduce) the contraction force (diameter reduction force) generated when the stent body 30 is expanded and indwelled compared to the contraction force when the stent body 30 is expanded and indwelled at a larger dimension (outer diameter) than the outer diameter of the base material 20. Therefore, it is possible to considerably decrease a recoil rate of the stent 10. It is also thus possible to utilize a stent manufacturing method to manufacture a stent 10 whose recoil rate is decreased.

The stent 10 has the connection portions 60 which are connected to each other in the axial direction of the stent body 30 and whose connecting forces decrease with the lapse of a predetermined period of time after the stent 10 indwells the living body (e.g., in a body lumen). Flexibility of the stent 10 can be improved in response to the decreased connecting force of the connection portions 60. Therefore, satisfactory followability inside the lumen is achieved.

The outer diameter D1 of the base material 20 is equal to or larger than the expansion start outer diameter D2 of the stent 10. Accordingly, the contraction force applied to the stent body 30 when the connecting force of the connection portions 60 decreases can be controlled to be lower than the contraction force of the stent configured so that the outer diameter D1 of the base material 20 is equal to or smaller than the expansion start outer diameter D2. Therefore, it is possible to decrease the recoil rate of the stent 10.

The connection portion 60 has the connection member 71 that includes the biodegradable material as described above. The connection member 71 makes it possible to decrease the connecting force over a lapse of time in accordance with the degradation of the biodegradable material. Therefore, it is possible to achieve satisfactory followability inside the body lumen.

When the connection member 71 that includes the biodegradable material starts to degrade, stress accumulated in the connection member 71 when the stent 10 is deformed with diameter expansion (i.e., is expanded radially outwardly) is released. A relatively strong contraction force can be applied to the stent body 30. In the stent 10 illustrated in FIG. 1(A), however, the outer diameter D1 of the base material 20 is equal to or larger than the expansion start outer diameter D2. The stress applied to the stent body 30 when the stent body 30 is expanded radially outward can thus be controlled to be low. Therefore, the contraction force applied when the connection member 71 starts to degrade can also be controlled to be low. This makes it possible to decrease the recoil rate of the stent 10.

The stent body 30 includes the strut 41, which has the helical portions 43 that extend in a helical shape around the axis of the stent body 30. The strut 41 is configured so that the connection portion 60 connects the adjacent helical portions 43 to each other in at least one location. The stent 10 can thus be relatively flexible, and the stent can possess desirable rigidity.

When the stent 10 is deformed with diameter expansion (i.e., the stent 10 is expanded radially outward from the compressed position on the outer surface of the balloon), stress is applied that twists the helical portion 43 formed in the stent body 30 in the axial direction and the circumferential direction. When the stent 10 is deformed with diameter expansion, the adjacent helical portions 43 are connected to each other by the connection portion 60. Accordingly, the stress is accumulated in the connection portion 60 via the helical portion 43, and the connection portion 60 restrains the helical portion 43 from being deformed. The stress accumulated in the connection portion 60 is released when the connecting force of the connection portion 60 decreases after the stent 10 is expanded radially outward to indwell the body lumen, and a relatively strong contraction force is applied to the helical portion 43. In the stent 10 described in this application, however, the outer diameter D1 of the base material 20 is equal to or larger than the expansion start outer diameter D2. Accordingly, the stress applied to the helical portion 43 when the stent body is deformed with diameter expansion can be controlled to be relatively low. The contraction force applied to the helical portion 43 when the connecting force of the connection portion 60 decreases can also be controlled to be low. That is, since the outer diameter D1 of the base material 20 is relatively large, a spread (rising) of a helical angle decreases when the stent body is deformed with diameter expansion. This minimizes the stress applied in a direction in which the helical angle is narrowed after the stent body indwells. As a result, it is possible to decrease the recoil rate.

A stent 10 in which the expansion start outer diameter D2 is 2.1 mm or larger when the stent indwells the living body may possess a further decreased recoil rate.

When a stent in which the expansion start outer diameter D2 is 3.0 mm or larger when the stent indwells the living body is used, it is possible to provide a stent 10 whose recoil rate is further decreased.

### APPLICATION EXAMPLE

Next, an application example is described in which the recoil rate of the above-described stent 10 is measured. The application example described below is an example of a stent disclosed in this application. The inventive stent is not limited to a configuration described below.

A stent 10 is prepared for the application example which satisfies the relationship of the outer diameter (D1) of the base material 20 the expansion start outer diameter (D2) of the stent 10. A cylindrical shaped pipe material formed of a cobalt-chromium alloy whose outer diameter D1 is 3.0 mm is used as the base material 20 of the stent 10. Laser processing (e.g., laser cuffing) is performed on the base material 20, thereby forming the strut 41 including the helical portions 43. The adjacent helical portions 43 are connected to each other via the connection portion 60. Polylactic acid is used for the connection member 71 included in the connection portion 60. The shape of the strut 41 and the configuration of the connection portion 60 are substantially the same as those which are illustrated in FIG. 1(B) described above.

A stent is prepared for a comparative example which satisfies the relationship of the outer diameter (D1) of the base material<the expansion start outer diameter (D2). A cylindrical shaped pipe material formed of a cobalt-chromium alloy whose outer diameter D1 is 2.0 mm is used as the base material of the stent. Other conditions of the stent according to the comparative example are the same as those of the stent 10 according to the application example.

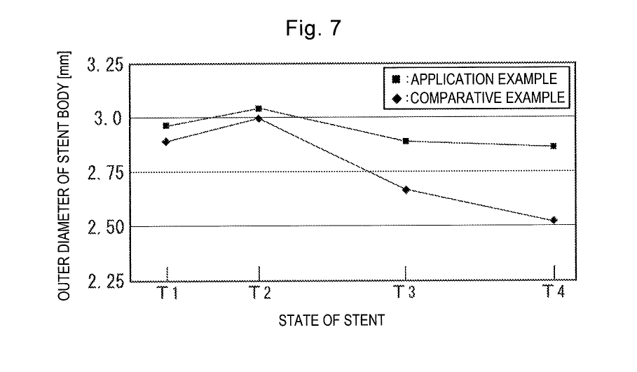

FIG. 7 illustrates a relationship between the dilating pressure of the balloon and the outer diameter of the stent body 30. T1 in FIG. 7 represents the outer diameter D2 of the stent body 30 when the balloon is dilated at the dilating start pressure (i.e., nominal pressure [atm]−2 [atm]). T2 in FIG. 7 represents the outer diameter of the stent body 30 when the balloon is dilated at the nominal pressure. T3 in FIG. 7 illustrates the outer diameter of the stent body 30 when the balloon is deflated and the diameter expansion using the balloon is released. T4 in FIG. 7 represents the outer diameter of the stent body 30 when the degraded connection member 71 weakens the connecting force between the helical portions 43.

The outer diameter of the stent body 30 of the application example (stent 10) is 2.934 mm at the time of T1, 3.045 mm at the time of T2, 2.892 mm at the time of T3, and 2.867 mm at the time of T4. The outer diameter of the stent body 30 is an average value of values measured at a plurality of optional locations in the axial direction of the stent 10 (average value of dimensions in the vicinity of the minimum outer diameter).

The recoil rate of the stent 10 is 5.0% at the time of T3, and is 5.8% at the time of T4. The recoil rate is a diameter reduction rate (outer diameter rate) of the outer diameter of the stent body 30 at the time of T3 and T4 with respect to the outer diameter of the stent body 30 at the time of T2.

The outer diameter of the stent body of the comparative example is 2.863 mm at the time of T1, 2.998 mm at the time of T2, 2.668 mm at the time of T3, and 2.523 mm at the time of T4. The recoil rate of the stent body is 11.1% at the time of T3, and is 15.8% at the time of T4.

Based on the results illustrated in FIG. 7, it is possible to confirm that both the recoil rates of the stent 10 (i.e., the recoil rate when the diameter expansion of the stent using the balloon is released and the recoil rate when the connection member 71 degrades) according to the application example are smaller than the recoil rates of the stent according to the comparative example. In other words, the stent 10 of the application example has a smaller recoil rate than the stent of the comparative example.

The inventive stent has been described in reference to the embodiment illustrated in FIGS. 1(A)-5(B). However, the stent can be appropriately modified based on the scope of claims and is not limited to only the configurations described above. For example, the above description relates to a stent that is caused to indwell after being deformed with diameter expansion by the dilated balloon.

The stent is not limited to the configuration in which the base material is a metal material. Any configuration may be adopted as long as the stent is configured to include an elastically deformable material to be capable of causing the recoil. For example, the base material of the stent may be configured to include a biodegradable polymer material.

The shape or the design (arrangement) of the strut of the stent, and the structure of the stent body are not limited to the forms described above in reference to the drawings. The shape and/or design of the strut of the stent can be appropriately changed as long as the linear portion is included in the strut. For example, various structures used for known stents (such as a structure having no connection portion added thereto, a structure of the strut having a shape other than the coil shape, and a structure of the strut having no bellow portion formed therein) are applicable to the stent disclosed in this application.

As long as the diameter (outer diameter, inner diameter, or both of these) of the base material has a dimension which is equal to or larger than the diameter (outer diameter, inner diameter, or both of these) of the stent body when the stent body is used after the dimension is expanded (i.e., the stent body has been expanded radially outward), a structure, a dimension, and a shape of each portion can be appropriately changed. The use of the additional member described in the embodiment can be omitted, or other members which are not particularly described in the embodiment can be additionally and appropriately used.

The detailed description above describes a stent and a method for manufacturing a stent. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A stent comprising:
a stent body comprising a strut formed from a cylindrical base material, the strut possessing a manufactured minimum outer diameter when the strut is formed from the cylindrical base material, the stent body extending in an axial direction and possessing a radial direction and a circumferential direction;
the stent body being configured to be compressed onto an outer surface of an expandable member of a catheter, the strut possessing a contracted outer diameter when the stent body is compressed onto the outer surface of the expandable member of the catheter;
the stent body being configured to be positioned in a living body while being compressed on the outer surface of the expandable member and expanded radially outward by applying a nominal pressure directed by a manufacturer within the expandable member to expand the expandable member in the radial direction;
the strut possessing an expanded outer diameter when the stent body is expanded by the expandable member at a pressure which is 2 atm lower than the nominal pressure, the expanded outer diameter of the strut being larger than the contracted outer diameter of the strut; and
the manufactured minimum outer diameter of the strut being equal to or larger than expanded outer diameter of the strut when the stent body is expanded by the expandable member at the pressure which is 2 atm lower than the nominal pressure.

2. The stent according to claim 1, wherein the manufactured minimum outer diameter of the strut is equal to or larger than a second expanded outer diameter of the strut when the expandable member is expanded at a second pressure which is 1 atm lower than the nominal pressure to expand the stent radially outwardly.

3. The stent according to claim 1, wherein
the cylindrical base material possesses an outer diameter and an inner diameter which are measured based on the manufactured minimum outer diameter of the strut,
the expanded outer diameter of the strut is equal to or smaller than the outer diameter of the base material, and
the strut possesses an expanded inner diameter when the stent body is expanded by the expandable member at a pressure which is 2 atm lower than the nominal pressure, the expanded inner diameter of the strut being equal to or smaller than the inner diameter of the base material.

4. The stent according to claim 1, further comprising:
connection portions that are connected to each other in the axial direction of the stent body, the connection portions decreasing a connecting force after a predetermined period of time elapses from when the stent indwells a living body.

5. The stent according to claim 4, wherein the connection portions comprise a connection member that includes a biodegradable material.

6. The stent according to claim 4, wherein
the strut comprises a helical portion which extends in a helical shape around the axial direction of the stent body, and
the connection portions connect adjacent helical portions to each other in at least one location.

7. The stent according to claim 3,
wherein the outer diameter of the base material and the outer diameter of the strut when the stent body is expanded by the expandable member at a pressure which is 2 atm lower than the nominal pressure are 2.1 mm or larger.

8. The stent according to claim 3, wherein the outer diameter of the base material is 3.0 mm or larger.

9. A stent kit, comprising:
the stent according to claim 1, and
a compliance chart provided by the manufacturer to correlate the nominal pressure with an intended outer diameter of the stent body.

10. The stent kit according to claim 9, wherein the compliance chart is provided on a sheet of paper.

11. A stent manufacturing method comprising:
forming a stent body by processing a base material having a cylindrical shape, the stent body comprising a strut, the stent body being configured to be compressed onto an outer surface of an expandable member of a catheter, the stent body further being configured to be positioned in a living body while being compressed on the outer surface of the expandable member and expanded radially outward by applying a nominal pressure directed by a manufacturer within the expandable member;
the base material having the cylindrical shape possessing an outer diameter;
the strut possessing an expanded outer diameter when the stent body is expanded in the living body by the expandable member at a pressure which is 2 atm lower than the nominal pressure; and
the outer diameter of the base material being equal to or larger than the expanded outer diameter of the strut when the expandable member is expanded at the pressure which is 2 atm lower than nominal pressure so as to radially outwardly expand the stent.

12. The stent manufacturing method according to claim 11, further comprising:
determining the nominal pressure to apply within the expandable member based on a diameter of a target body lumen; and
selecting an outer diameter of the base material based on the diameter of the target body lumen.

13. The stent manufacturing method according to claim 11, wherein
the forming of the stent body by processing the base material comprises cutting.

14. The stent manufacturing method according to claim 13, wherein the cutting is one of laser cutting, electric discharge machining, or chemical etching.

15. The stent manufacturing method according to claim 11, further comprising:
coating an outer surface of the strut with a drug after the forming of the stent body.

16. A stent manufacturing method comprising:
determining a target outer diameter for a stent based on a diameter of a target body lumen in a living body;
selecting a cylindrical base material with an outer diameter larger than the target outer diameter for the stent;
forming a stent body by cutting a strut out of the cylindrical base material, the stent body being configured to be compressed onto an outer surface of an expandable member of a catheter, the stent body further being configured to be moved to the target body lumen in the living body while being compressed on the outer surface of the expandable member and then expanded radially outward by applying a nominal pressure directed by a manufacturer within the expandable member;
the strut possessing an expanded outer diameter when the stent body is expanded in the living body by the expandable member at a pressure which is 2 atm lower than the nominal pressure; and
the outer diameter of the cylindrical base material being equal to or larger than the expanded outer diameter of the strut when the expandable member is expanded at the pressure which is 2 atm lower than nominal pressure so as to radially outwardly expand the stent.

17. The stent manufacturing method according to claim 16, wherein the cutting of the strut out of the cylindrical base material comprises cutting at least one helically-shaped portion out of the cylindrical base material so that the strut comprises at least one helical portion.

18. The stent manufacturing method according to claim 16, further comprising:
measuring the outer diameter of the strut after the stent body is formed and before the stent is inserted into the living body.

19. The stent manufacturing method according to claim 16, wherein the cutting of the strut out of the cylindrical base material is by one of laser cutting, electric discharge machining, or chemical etching.

20. The stent manufacturing method according to claim 16, further comprising:
coating an outer surface of the strut with a drug after the forming of the stent body.
